(19) United States
(12) Patent Application Publication
(10) Pub. No.: US 2024/0343624 A1
(43) Pub. Date: Oct. 17, 2024

(54) MICROORGANISMS FOR TREATMENT OF HYDROCARBONS OR OIL

(71) Applicant: MICROBIAL DISCOVERY GROUP, LLC, Franklin, WI (US)

(72) Inventors: Michael R. KING, Oak Creek, WI (US); Sona SON, Franklin, WI (US); Megan DUERSTELER, Franklin, WI (US)

(21) Appl. No.: 18/682,226

(22) PCT Filed: Aug. 9, 2022
(86) PCT No.: PCT/US2022/039787

§ 371 Date: Feb. 8, 2024

Related U.S. Application Data
(60) Provisional application No. 63/230,983, filed on Aug. 9, 2021.

(51) Int. Cl. C02F 3/34; B08B 7/00; B09C 1/10; C02F 101/32; C12N 1/20; C12R 1/07; C12R 1/10

(52) U.S. Cl. CPC C02F 3/344; B08B 7/0014; B09C 1/10; C02F 3/342; C02F 3/348; C12N 1/205; B09C 2101/00; C02F 2101/32; C12R 2001/07; C12R 2001/10

(57) ABSTRACT

The invention relates to compositions comprising microbial strains and methods for use of the compositions in removing hydrocarbons from water and/or soil. More particularly, the invention relates to compositions of isolated Bacillus strains selected from the group consisting of isolated Bacillus strains Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains, and combinations thereof, and methods for use of these strains for removing hydrocarbons from water and/or soil. The invention also relates to a method of removing oil in a cleaning application as described herein.

### CROSS-REFERENCE TO RELATED APPLICATIONS

This application claims the benefit under 35 USC § 119 (e) of U.S. Provisional Application Ser. No. 63/230,983, filed on Aug. 9, 2021, the entire disclosure of which is incorporated herein by reference.

### FIELD OF THE DISCLOSURE

The invention relates to compositions comprising microbial strains and methods for use of the compositions in removing hydrocarbons from water and/or soil. More particularly, the invention relates to compositions of isolated Bacillus strains selected from the group consisting of isolated Bacillus strains Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains, and combinations thereof, and methods for use of these strains for removing hydrocarbons from water and/or soil. The invention also relates to a method of removing oil in a cleaning application as described herein.

### BACKGROUND AND SUMMARY OF THE INVENTION

Crude oil refining and other processes can result in environmental total petroleum hydrocarbon (TPH) contamination—a potential safety risk to humans, animals, and plants. Contaminated soil and wastewater may be remediated through bioaugmentation. Environments with high levels of TPH contamination contain a variety of aromatic and aliphatic hydrocarbons in the gas, diesel, and oil ranges, thus an effective remediation treatment needs to be effective in degrading multiple types of hydrocarbons. The long-chain hydrocarbons are of particular interest, as they are the slowest and most difficult to degrade through bioaugmentation. The inventors have surprisingly developed a multi-strain Bacillus bioremediation product that is able to metabolize gas range organics (GRO), diesel range organics (DRO), and oil range organics (ORO).

In one embodiment, a method of treating water and/or soil by contacting the water and/or the soil with a Bacillus strain to remove a hydrocarbon is provided. The method comprises contacting the water and/or the soil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the hydrocarbon.

In another embodiment, a commercial package is provided. The commercial package comprises isolated Bacillus strains consisting of Bacillus strains Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains.

In another embodiment, a method of removing oil in a cleaning application by contacting the oil with a Bacillus strain to remove the oil is provided. The method comprises contacting the oil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the oil.

The various embodiments described in the numbered clauses below are applicable to any of the embodiments described in this “SUMMARY” section and the sections of the patent application titled “DETAILED DESCRIPTION OF ILLUSTRATIVE EMBODIMENTS” or “EXAMPLES” or in the “CLAIMS” appended to this application:
1. A method of treating water and/or soil by contacting the water and/or the soil with a Bacillus strain to remove a hydrocarbon, the method comprising contacting the water and/or the soil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the hydrocarbon.
2. The method of clause 1 wherein water is treated.
3. The method of clause 1 wherein soil is treated.
4. The method of any one of clauses 1 to 3 wherein the hydrocarbon is a short-chain to medium-chain hydrocarbon.
5. The method of any one of clauses 1 to 4 wherein the hydrocarbon is selected from the group consisting of toluene, benzene, kerosene, and combinations thereof.
6. The method of any one of clauses 1 to 4 wherein the hydrocarbon is selected from the group consisting of a gas range organic (GRO), a diesel range organic (DRO), and an oil range organic (ORO).
7. The method of any one of clauses 1 to 6 wherein the hydrocarbon is a petroleum hydrocarbon.
8. The method of any one of clauses 1 to 3 or 6 to 7 wherein the hydrocarbon is a long-chain hydrocarbon.
9. The method of any one of clauses 1 to 8 wherein the Bacillus strain produces an enzyme selected from the group consisting of an oxidoreductase, a hydrolase, a transferase, a lyase, an isomerase, a ligase, a peroxidase, a laccase, an esterase, an amylase, a protease, a xylanase, a lipase, a cellulase, an oxygenase, a reductase, an oxidase, a hydroxylase, a dehydrogenase, and combinations thereof.
10. The method of any one of clauses 1 to 9 further comprising treating the water and/or the soil with another Bacillus strain.
11. The method of any one of clauses 1 to 10 wherein the effective amount of the Bacillus strain is about 1.0×10² CFU/gram of the water and/or the soil to about 1.0×10⁶ CFU/gram of the water and/or the soil.
12. The method of any one of clauses 1 to 10 wherein the effective amount of the Bacillus strain is about 1.0×10² CFU/gram of the water and/or the soil to about 1.0×10⁴ CFU/gram of the water and/or the soil.
13. The method of any one of clauses 1 to 10 wherein the effective amount of the Bacillus strain is about 1.0×10² CFU/gram of the water and/or the soil to about 1.0×10³
CFU/gram of the water and/or the soil.
14. The method of any one of clauses 1 to 13 further comprising contacting the water and/or the soil with an exogenous enzyme.
15. The method of any one of clauses 1 to 14 wherein the strain administered is Bacillus strain Bacillus licheniformis MDG-1000 (NRRL No. B-67888), or a strain having all of the identifying characteristics of Bacillus licheniformis MDG-1000 (NRRL No. B-67888).
16. The method of any one of clauses 1 to 14 wherein the strain administered is Bacillus strain Bacillus amyloliquefaciens MDG 1607 (NRRL No. B-67666), or a strain having all of the identifying characteristics of Bacillus amyloliquefaciens strain MDG1607 (NRRL No. B-67666).
17. The method of any one of clauses 1 to 14 wherein the strain administered is Bacillus strain Bacillus pumilus MDG-1047 (NRRL No. B-67891), or a strain having all of the identifying characteristics of Bacillus pumilus MDG-1047 (NRRL No. B-67891).
18. The method of any one of clauses 1 to 14 wherein the strain administered is Bacillus strain Bacillus licheniformis MDG-1000 (NRRL No. B-67888).
19. The method of any one of clauses 1 to 14 wherein the strain administered is Bacillus strain Bacillus amyloliquefaciens MDG1607 (NRRL No. B-67666).
20. The method of any one of clauses 1 to 14 wherein the strain administered is Bacillus strain Bacillus pumilus MDG-1047 (NRRL No. B-67891).
21. A commercial package comprising isolated Bacillus strains wherein the Bacillus strains consist of Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains.
22. The commercial package of clause 21 wherein the Bacillus strains cause degradation of a hydrocarbon in water and/or soil.
23. The commercial package of clause 21 or 22 wherein the Bacillus strains are in the form of a concentrate.
24. The commercial package of clause 21 or 22 wherein the Bacillus strains are in the form of a superconcentrate.
25. The commercial package of any one of clauses 21 to 24 wherein the Bacillus strains are in dry form.
26. The commercial package of any one of clauses 21 to 24 wherein the Bacillus strains are in a liquid form.
27. The commercial package of any one of clauses 21 to 26 further comprising a carrier for the Bacillus strains.
28. The commercial package of any one of clauses 21 to 27 wherein the commercial package comprises a bag.
29. The commercial package of clause 28 wherein the bag is a plastic bag.
30. The commercial package of any one of clauses 21 to 29 further comprising instructions for use of the Bacillus strains.
31. The commercial package of any one of clauses 21 to 30 comprising a 20-pound bag.
32. The commercial package of any one of clauses 21 to 30 comprising a 50-pound bag.
33. The commercial package of any one of clauses 21 to 25 or 27 to 32 in powder form.
34. The commercial package of any one of clauses 21 to 33 comprising a container for commercial use wherein the container comprises plastic.
35. The commercial package of any one of clauses 21 to 28 or 30 to 33 comprising a container for commercial use wherein the container comprises paper.
36. The commercial package of any one of clauses 21 to 35 further comprising a binder.
37. The commercial package of clause 36 wherein the binder is selected from the group consisting of clay, yeast cell wall components, aluminum silicate, and glucan, or combinations thereof.
38. The commercial package of clause 27 wherein the carrier is selected from the group consisting of a salt, mineral oil, a dextrin, whey, sugar, sucrose, limestone, yeast culture, dried starch, sodium silico aluminate, silicon dioxide, polypropylene glycol, polysorbate 80, vegetable oil, water, rice hulls, and combinations thereof.
39. A method of removing oil in a cleaning application by contacting the oil with a Bacillus strain to remove the oil, the method comprising contacting the oil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the oil.
40. The method of clause 39 wherein the cleaning application is an industrial cleaning application.
41. The method of clause 39 wherein the oil is from an oil stain on concrete.
42. The method of clause 39 wherein the oil is on metal.
43. The method of clause 42 wherein the metal is part of a piece of equipment.
44. The method of clause 42 wherein the metal is part of a vehicle.

### BRIEF DESCRIPTION OF THE DRAWINGS

FIG. 1 shows increased bacterial activity after 36 hours in a soil mixture spiked with benzene, indicating the benzene was metabolized as a carbon source (p<0.10, Two sample t-test) by the Bacillus strain combination described in this application.

FIG. 2 shows increased bacterial activity after 36 hours in a soil mixture spiked with toluene, indicating the toluene was metabolized as a carbon source (p<0.05, Two sample t-test) by the Bacillus strain combination described in this application.

FIG. 3 shows increased bacterial activity after 36 hours in a soil mixture spiked with kerosene, indicating the kerosene was metabolized as a carbon source (p<0.05, Two sample t-test) by the Bacillus strain combination described in this application.

FIG. 4 shows increased bacterial activity after 6 days in media with paraffin wax as the sole carbon source, indicating the paraffin wax was metabolized as a carbon source (p<0.05, Two sample t-test) by the Bacillus strain combination described in this application.

FIG. 5 shows a photograph of a gel displaying RAPD-PCR profiles for Bacillus strain 1000. The leftmost and rightmost lanes, for each group of eight lanes, include markers.

FIG. 6 shows a photograph of a gel displaying RAPD-PCR profiles for Bacillus strain 1047. The leftmost and rightmost lanes, for each group of eight lanes, include markers.

FIG. 7 shows a photograph of a gel displaying RAPD-PCR profiles for Bacillus strain 1607. The leftmost and rightmost lanes include markers.

### DETAILED DESCRIPTION OF ILLUSTRATIVE EMBODIMENTS

The invention relates to compositions comprising microbial strains and methods for use of the compositions in removing hydrocarbons from water and/or soil. More particularly, the invention relates to compositions of isolated Bacillus strains selected from the group consisting of isolated Bacillus strains Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains, and combinations thereof, and methods for use of these strains for removing hydrocarbons from water and/or soil. The invention also relates to a method of removing oil in a cleaning application as described herein.

The various embodiments described in the numbered clauses below are applicable to any of the embodiments described in this “DETAILED DESCRIPTION OF ILLUSTRATIVE EMBODIMENTS” section and the sections of the patent application titled “SUMMARY” or “EXAMPLES” or in the “CLAIMS” appended to this application:
1. A method of treating water and/or soil by contacting the water and/or the soil with a Bacillus strain to remove a hydrocarbon, the method comprising contacting the water and/or the soil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the hydrocarbon.
2. The method of clause 1 wherein water is treated.
3. The method of clause 1 wherein soil is treated.
4. The method of any one of clauses 1 to 3 wherein the hydrocarbon is a short-chain to medium-chain hydrocarbon.
5. The method of any one of clauses 1 to 4 wherein the hydrocarbon is selected from the group consisting of toluene, benzene, kerosene, and combinations thereof.
6. The method of any one of clauses 1 to 4 wherein the hydrocarbon is selected from the group consisting of a gas range organic (GRO), a diesel range organic (DRO), and an oil range organic (ORO).
7. The method of any one of clauses 1 to 6 wherein the hydrocarbon is a petroleum hydrocarbon.
8. The method of any one of clauses 1 to 3 or 6 to 7 wherein the hydrocarbon is a long-chain hydrocarbon.
9. The method of any one of clauses 1 to 8 wherein the Bacillus strain produces an enzyme selected from the group consisting of an oxidoreductase, a hydrolase, a transferase, a lyase, an isomerase, a ligase, a peroxidase, a laccase, an esterase, an amylase, a protease, a xylanase, a lipase, a cellulase, an oxygenase, a reductase, an oxidase, a hydroxylase, a dehydrogenase, and combinations thereof.
10. The method of any one of clauses 1 to 9 further comprising treating the water and/or the soil with another Bacillus strain.
11. The method of any one of clauses 1 to 10 wherein the effective amount of the Bacillus strain is about 1.0×10² CFU/gram of the water and/or the soil to about 1.0×10⁶ CFU/gram of the water and/or the soil.
12. The method of any one of clauses 1 to 10 wherein the effective amount of the Bacillus strain is about 1.0×10² CFU/gram of the water and/or the soil to about 1.0×10⁴ CFU/gram of the water and/or the soil.
13. The method of any one of clauses 1 to 10 wherein the effective amount of the Bacillus strain is about 1.0×10² CFU/gram of the water and/or the soil to about 1.0×10³ CFU/gram of the water and/or the soil.
14. The method of any one of clauses 1 to 13 further comprising contacting the water and/or the soil with an exogenous enzyme.
15. The method of any one of clauses 1 to 14 wherein the strain administered is Bacillus strain Bacillus licheniformis MDG-1000 (NRRL No. B-67888), or a strain having all of the identifying characteristics of Bacillus licheniformis MDG-1000 (NRRL No. B-67888).
16. The method of any one of clauses 1 to 14 wherein the strain administered is Bacillus strain Bacillus amyloliquefaciens MDG 1607 (NRRL No. B-67666), or a strain having all of the identifying characteristics of Bacillus amyloliquefaciens strain MDG1607 (NRRL No. B-67666).
17. The method of any one of clauses 1 to 14 wherein the strain administered is Bacillus strain Bacillus pumilus MDG-1047 (NRRL No. B-67891), or a strain having all of the identifying characteristics of Bacillus pumilus MDG-1047 (NRRL No. B-67891).
18. The method of any one of clauses 1 to 14 wherein the strain administered is Bacillus strain Bacillus licheniformis MDG-1000 (NRRL No. B-67888).
19. The method of any one of clauses 1 to 14 wherein the strain administered is Bacillus strain Bacillus amyloliquefaciens MDG1607 (NRRL No. B-67666).
20. The method of any one of clauses 1 to 14 wherein the strain administered is Bacillus strain Bacillus pumilus MDG-1047 (NRRL No. B-67891).
21. A commercial package comprising isolated Bacillus strains wherein the Bacillus strains consist of Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains.
22. The commercial package of clause 21 wherein the Bacillus strains cause degradation of a hydrocarbon in water and/or soil.
23. The commercial package of clause 21 or 22 wherein the Bacillus strains are in the form of a concentrate.
24. The commercial package of clause 21 or 22 wherein the Bacillus strains are in the form of a superconcentrate.
25. The commercial package of any one of clauses 21 to 24 wherein the Bacillus strains are in dry form.
26. The commercial package of any one of clauses 21 to 24 wherein the Bacillus strains are in a liquid form.
27. The commercial package of any one of clauses 21 to 26 further comprising a carrier for the Bacillus strains.
28. The commercial package of any one of clauses 21 to 27 wherein the commercial package comprises a bag.
29. The commercial package of clause 28 wherein the bag is a plastic bag.
30. The commercial package of any one of clauses 21 to 29 further comprising instructions for use of the Bacillus strains.
31. The commercial package of any one of clauses 21 to 30 comprising a 20-pound bag.
32. The commercial package of any one of clauses 21 to 30 comprising a 50-pound bag.
33. The commercial package of any one of clauses 21 to 25 or 27 to 32 in powder form.
34. The commercial package of any one of clauses 21 to 33 comprising a container for commercial use wherein the container comprises plastic.
35. The commercial package of any one of clauses 21 to 28 or 30 to 33 comprising a container for commercial use wherein the container comprises paper.
36. The commercial package of any one of clauses 21 to 35 further comprising a binder.
37. The commercial package of clause 36 wherein the binder is selected from the group consisting of clay, yeast cell wall components, aluminum silicate, and glucan, or combinations thereof.
38. The commercial package of clause 27 wherein the carrier is selected from the group consisting of a salt, mineral oil, a dextrin, whey, sugar, sucrose, limestone, yeast culture, dried starch, sodium silico aluminate, silicon dioxide, polypropylene glycol, polysorbate 80, vegetable oil, water, rice hulls, and combinations thereof.
39. A method of removing oil in a cleaning application by contacting the oil with a Bacillus strain to remove the oil, the method comprising contacting the oil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the oil.
40. The method of clause 39 wherein the cleaning application is an industrial cleaning application.
41. The method of clause 39 wherein the oil is from an oil stain on concrete.
42. The method of clause 39 wherein the oil is on metal.
43. The method of clause 42 wherein the metal is part of a piece of equipment.
44. The method of clause 42 wherein the metal is part of a vehicle.

In various water and/or soil treatment embodiments, the water and/or the soil that is treated to remove a hydrocarbon using the Bacillus strains described herein can be selected from the group consisting of water, industrial wastewater, wastewater, residential wastewater, soil, soil waste, contaminated groundwater, and leachate from waste.

In another embodiment, the Bacillus strains described herein can be used to remove oil in industrial cleaning applications (e.g., cleaning oil stains from concrete (e.g., a driveway) or cleaning oil from metal (e.g., from equipment or vehicles). In this embodiment, as used herein, “removal”, “removing”, or “removed” mean to remove and/or to degrade a portion of the oil, or to remove and/or degrade all of the oil.

In various embodiments of the water and/or soil treatment methods described herein, the Bacillus strains (e.g., Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains) can have an effect, including but not limited to, effects selected from the group consisting of removal of a hydrocarbon, removal of a short-chain hydrocarbon, removal of a long-chain hydrocarbon, removal of a medium-chain hydrocarbon, removal of a petroleum hydrocarbon, removal of an aliphatic hydrocarbon, removal of an aromatic hydrocarbon, remediation of water and/or soil, removal of a gas range organic (GRO), removal of a diesel range organic (DRO), and removal of an oil range organic (ORO) from water and/or soil. As used herein, “removal”, “removing”, or “removed” mean to remove and/or to degrade a portion of the hydrocarbons in water and/or soil, to remove and/or degrade all of the hydrocarbons in water and/or soil, to cause the hydrocarbons to be converted to an inactivated form in water and/or soil, or to reduce the activity of hydrocarbons in water and/or soil.

In one aspect, the short-chain or medium-chain hydrocarbon can be selected from the group consisting of toluene, benzene, kerosene, and combinations thereof. In other embodiments, the hydrocarbons can be from about C6 to about C20.

In any water and/or soil treatment embodiments described herein, the Bacillus strains may be added to water and/or soil alone or in any combination, or can be in the form of any composition described herein so that the strains are alone or in any combination in the compositions described herein. The Bacillus strains described herein can also be used in combination with other microbial strains, including other Bacillus strains. In another embodiment, the additional Bacillus strain can be selected from the group consisting of a strain of Bacillus subtilis, Bacillus amyloliquefaciens, Bacillus licheniformis, Bacillus pumilus, Bacillus mojavensis, Bacillus methylotrophicus, or other Bacillus strains, and combinations thereof.

In one embodiment of the invention, an effective amount of the Bacillus strain can be added to water and/or soil to cause an effect, including but not limited to, effects selected from the group consisting of removal of a hydrocarbon, removal of a short-chain hydrocarbon, removal of a long-chain hydrocarbon, removal of a medium-chain hydrocarbon, removal of a petroleum hydrocarbon, removal of an aliphatic hydrocarbon, removal of an aromatic hydrocarbon, remediation of water and/or soil, removal of a gas range organic (GRO), removal of a diesel range organic (DRO), and removal of an oil range organic (ORO) from water and/or soil. An “effective amount” is an amount that causes any of the effects described in this paragraph.

The compositions described herein can be used to treat water and/or soil for any period of time that is effective to remove a hydrocarbon. For example, in one embodiment treatment of water and/or soil can occur daily. The time periods for treatment of water and/or soil are non-limiting and it should be appreciated that any time period or treatment schedule determined to be effective to remove hydrocarbons may be used.

In any of the embodiments described herein, the Bacillus strain can produce an enzyme selected from the group consisting of an oxidoreductase, a hydrolase, a transferase, a lyase, an isomerase, a ligase, a peroxidase, a laccase, an esterase, an amylase, a protease, a xylanase, a lipase, a cellulase, an oxygenase, a reductase, an oxidase, a hydroxylase, a dehydrogenase, and combinations thereof.

In another illustrative embodiment, one or more enzymes may be added to the water and/or the soil along with the Bacillus strains described herein. In various embodiments, the exogenous enzymes that may be added include an exogenous enzyme selected from the group consisting of an oxidoreductase, a hydrolase, a transferase, a lyase, an isomerase, a ligase, a peroxidase, a laccase, an esterase, an amylase, a protease, a xylanase, a lipase, a cellulase, an oxygenase, a reductase, an oxidase, a hydroxylase, a dehydrogenase, and combinations thereof.

In various illustrative embodiments of the water and/or soil treatment methods, Bacillus strain Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and/or a strain having all of the identifying characteristics of these strains can be added to the water and/or the soil in an effective amount of about 1.0×10² CFU/gram of the water and/or the soil to about 1.0×10⁶ CFU/gram of the water and/or the soil, in an effective amount of about 1.0×10² CFU/gram of the water and/or the soil to about 1.0×10⁴ CFU/gram of the water and/or the soil, in an effective amount of about 1.0×10² CFU/gram of the water and/or the soil to about 1.0×10³ CFU/gram of the water and/or the soil, in an effective amount of about 1.0×10² CFU/gram of the water and/or the soil to about 5.0×10¹² CFU/gram of the water and/or the soil, at an effective amount of about 1.0×10² CFU/gram of the water and/or the soil to about 1.0×10⁷ CFU/gram of the water and/or the soil, in an effective amount of about 1.0×10² CFU/gram of the water and/or the soil to about 1.0×10⁸ CFU/gram of the water and/or the soil, in an effective amount of about 1.0×10² CFU/gram of the water and/or the soil to about 1.0×10⁹ CFU/gram of the water and/or the soil, in an effective amount of about 1.0×10² CFU/gram of the water and/or the soil to about 1.0×10¹⁰ CFU/gram of the water and/or the soil, or in an effective amount of about 1.0×10² CFU/gram of the water and/or the soil to about 1.0×10¹¹ CFU/gram of the water and/or the soil. In other embodiments, the Bacillus strain can be added to the water and/or the soil at an effective amount of greater than about 1.0×10² CFU/gram of the water and/or the soil, at an effective amount of greater than about 1.0×10³ CFU/gram of the water and/or the soil, at an effective amount of greater than about 1.1×10³ CFU/gram of the water and/or the soil, at an effective amount of greater than about 1.25×10³ CFU/gram of the water and/or the soil, at an effective amount of greater than about 1.5×10³ CFU/gram of the water and/or the soil, at an effective amount of greater than about 1.75×10³ CFU/gram of the water and/or the soil, at an effective amount of greater than about 1.0×10⁴ CFU/gram of the water and/or the soil, at an effective amount of greater than about 2.0×10⁴ CFU/gram of the water and/or the soil, at an effective amount of greater than about 3.0×10⁴ CFU/gram of the water and/or the soil, at an effective amount of greater than about 4.0×10⁴ CFU/gram of the water and/or the soil, at an effective amount of greater than about 5.0×10⁴ CFU/gram of the water and/or the soil, at an effective amount of greater than about 6.0×10⁴ CFU/gram of the water and/or the soil, at an effective amount of greater than about 7.0×10⁴ CFU/gram of the water and/or the soil, at an effective amount of greater than about 8.0×10⁴ CFU/gram of the water and/or the soil, at an effective amount of greater than about 1.0×10⁵ CFU/gram of the water and/or the soil, at an effective amount of greater than about 1.0×10⁶ CFU/gram of the water and/or the soil, at an effective amount of greater than about 1.0×10⁷ CFU/gram of the water and/or the soil, at an effective amount of greater than about 1.0×10⁸ CFU/gram of the water and/or the soil, at an effective amount of greater than about 1.0×10⁹ CFU/gram of the water and/or the soil, at an effective amount of greater than about 1.0×10¹⁰ CFU/gram of the water and/or the soil, at an effective amount of greater than about 1.0×10¹¹ CFU/gram of the water and/or the soil, or at an effective amount of greater than about 1.0×10¹² CFU/gram of the water and/or the soil. In another embodiment, any of the amounts described herein can be in CFU/ml of water in embodiments where the strains are added to any type of water or wastewater to treat the water or wastewater.

In one aspect, these strains can be added to water and/or the soil alone or in combination. In one embodiment, the strains are added to water and/or soil in combination in a single composition. In another embodiment, the strains are added to water and/or soil in combination in separate compositions.

In any embodiments described herein, the Bacillus strain Bacillus licheniformis MDG-1000 (NRRL No. B-67888), Bacillus pumilus MDG-1047 (NRRL No. B-67891), Bacillus amyloliquefaciens MDG1607 (NRRL No. B-67666), or a strain having all of the identifying characteristics of these strains can be used in accordance with the methods and compositions described herein. Bacillus strains Bacillus licheniformis MDG-1000 (NRRL No. B-67888), and Bacillus pumilus MDG-1047 (NRRL No. B-67891), were deposited on Nov. 26, 2019 at the Agricultural Research Service Culture Collection (NRRL), National Center for Agricultural Utilization Research, Agricultural Research Service, USDA, 1815 North University Street, Peoria, Illinois 61604-3999, and were given accession numbers B-67888 and B-67891, respectively. Bacillus strain Bacillus amyloliquefaciens MDG1607 (NRRL No. B-67666) was deposited on Aug. 9, 2018 at the Agricultural Research Service Culture Collection (NRRL), National Center for Agricultural Utilization Research, Agricultural Research Service, USDA, 1815 North University Street, Peoria, Illinois 61604-3999, and was given accession number B-67666. The deposits were made under the provisions of the Budapest Treaty on the International Recognition of the Deposit of Microorganisms for the Purposes of Patent Procedure.

As used herein “a strain having all of the identifying characteristics of” Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666) can be a mutant strain having all of the identifying characteristics of Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666) (e.g., having a DNA fingerprint based on DNA analysis that corresponds to the DNA fingerprint of Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666), enzyme activities that correspond to Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666), antimicrobial activity that corresponds to Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666), antibiotic sensitivity or tolerance profiles that correspond to Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666), or combinations of these identifying characteristics). In alternate embodiments, the mutation can be a natural mutation, or a genetically engineered mutation. In another embodiment, “a strain having all of the identifying characteristics of” Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666) can be a strain, for example, produced by isolating one or more plasmids from Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666), and introducing the one or more plasmids into another bacterium, such as another Bacillus strain, as long as the one or more plasmids contain DNA that provides the identifying characteristics of Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666) (e.g., having a DNA fingerprint based on DNA analysis that corresponds to the DNA fingerprint of Bacillus strain Bacillus licheniformis MDG-1000 (NRRL No. B-67888), Bacillus pumilus MDG-1047 (NRRL No. B-67891), or Bacillus amyloliquefaciens MDG1607 (NRRL No. B-67666)). In this application, when the phrase “and strains having all of the identifying characteristics of these strains” is used in reference to a list of strains, this phrase means that the additional strains among “strains having all of the identifying characteristics of these strains” each has all of the identifying characteristics of any one strain in the list.

In an additional embodiment of the invention, compositions are provided comprising isolated Bacillus strains wherein the strains consist of Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains.

In various embodiments, the Bacillus strains in the commercial package can causes an effect selected from the group consisting of removal of a hydrocarbon, removal of a short-chain hydrocarbon, removal of a long-chain hydrocarbon, removal of a medium-chain hydrocarbon, removal of a petroleum hydrocarbon, removal of an aliphatic hydrocarbon, removal of an aromatic hydrocarbon, remediation of water and/or soil, removal of a gas range organic (GRO), removal of a diesel range organic (DRO), and removal of an oil range organic (ORO) from water and/or soil.

In one illustrative aspect, in the commercial package Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and/or strains having all of the identifying characteristics of these strains can be in the form of a concentrate (e.g., about 1×10⁸ to about 5×10⁹ CFU/g) or a superconcentrate (e.g., about 1×10¹⁰ to about 5×10¹² CFU/g). In another embodiment, Bacillus strain Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and/or strains having all of the identifying characteristics of these strains in a composition in the commercial package, can be in a dry form (e.g., a powder or freeze-dried form), a liquid form, in the form of a gel, in the form of a paste, or any other suitable form.

In another illustrative embodiment, the commercial package comprising Bacillus strains consisting of Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains, can further comprise a carrier for the Bacillus strain. The carrier can be selected from the group consisting of a salt, mineral oil, a dextrin (e.g., maltodextrin), whey, sugar, sucrose, limestone, yeast culture, dried starch, sodium silico aluminate, silicon dioxide, polypropylene glycol, polysorbate 80, vegetable oil, water, rice hulls, and combinations thereof. In another embodiment, the carrier can be any suitable carrier known in the art.

In another embodiment, the commercial package comprising Bacillus strains consisting of Bacillus strain Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains can further comprise a binder such as clay, yeast cell wall components, aluminum silicate, glucan, or other known binders. In another embodiment, the commercial package can further comprise inorganic/organic binders, essential oils, and/or organic acids.

In yet other embodiments, the commercial package comprising Bacillus strains consisting of Bacillus strain Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains can be in a container for commercial use. In various embodiments the container can be, for example, a bag (e.g., a 20-pound bag, a 50-pound bag, a 2-ounce bag, a 1-pound bag, or a 1-kilogram bag), a pouch, a drum, a bottle, or a box. In illustrative aspects, the container for the commercial package can comprise plastic, metal, foil, paper, fiber, or cardboard (e.g., a plastic pail, a paper bag, a foil bag, a fiber drum, etc.). In yet another embodiment, the commercial package can further comprise instructions for use of the Bacillus strains.

The following EXAMPLES provide various additional illustrative aspects of the invention described herein and are not intended to be limiting in any way.

### Example 1

### Metabolism of Benzene, Toluene, and Kerosene

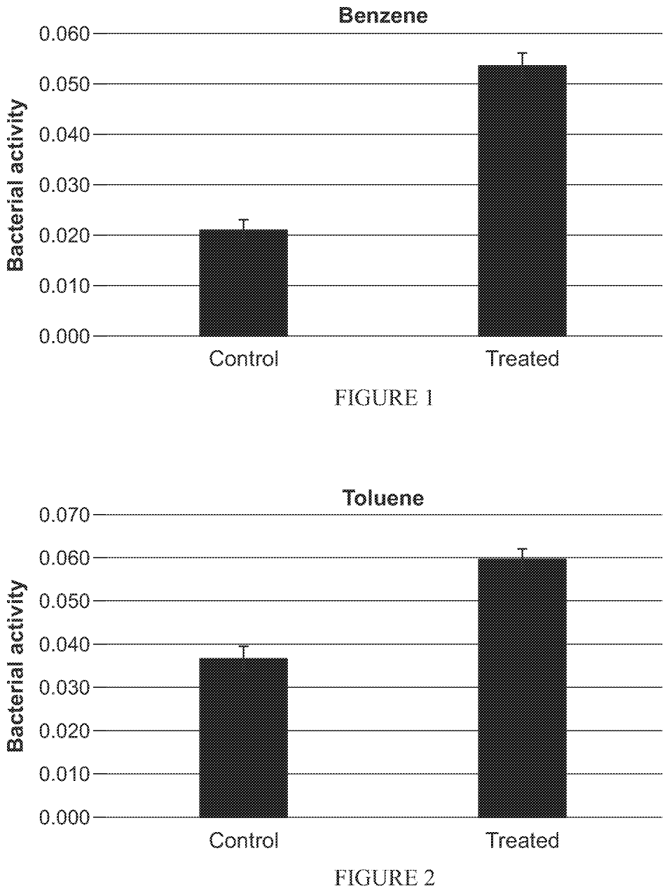

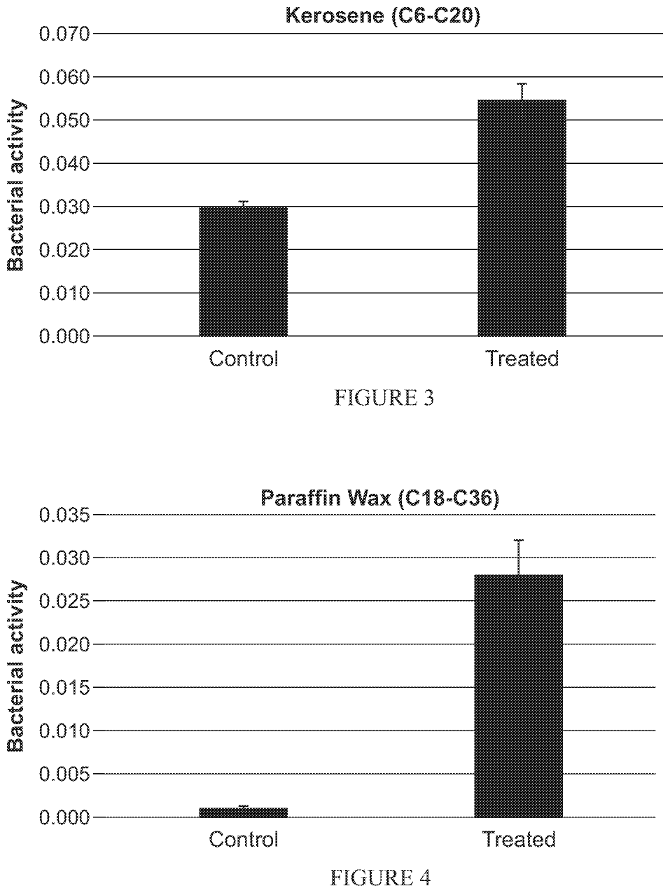

The Bacillus combination was tested for its ability to digest benzene, toluene, and kerosene, which were chosen as representative of short to medium chain hydrocarbons. The chemicals benzene (C6) and toluene (C7) are aromatic compounds within GRO. Kerosene contains a mixture of hydrocarbons from C6 to C20, which fall primarily within DRO. A soil slurry was created by mixing 1% soil by weight in water. The mixture was spiked with 4000 ppm of benzene, toluene, or kerosene and treated with a Bacillus combination which includes strains Bacillus pumilus MDG-1047 (NRRL B-67891), Bacillus licheniformis MDG-1000 (NRRL B-67888), and Bacillus amyloliquefaciens MDG-1607 (NRRL B-67666). Bacterial growth was measured by using a spectrophotometer over 36 hours. The hydrocarbon was the primary carbon source, therefore an increase in bacterial growth would indicate metabolism of the hydrocarbons. The Bacillus combination increased bacterial activity compared to the control in the soil mixtures spiked with benzene, toluene, and kerosene, indicating the Bacillus combination was metabolizing the hydrocarbons (FIGS. 1, 2, and 3).

### Example 2

### Metabolism of Paraffin Wax

The Bacillus combination was tested for its ability to digest paraffin wax. Paraffin wax contains a mixture of hydrocarbons from C18 to C36, which fall primarily within ORO. A 5 g piece of paraffin wax was placed in a carbon-free minimal media and treated with a Bacillus combination which includes strains Bacillus pumilus MDG-1047 (NRRL B-67891), Bacillus licheniformis MDG-1000 (NRRL B-67888), and Bacillus amyloliquefaciens MDG-1607 (NRRL B-67666). Bacterial growth was measured by using a spectrophotometer over 6 days. Paraffin wax was the sole available carbon source, therefore an increase in bacterial growth would indicate metabolism of paraffin wax. The Bacillus product increased bacterial activity compared to the control, indicating the Bacillus combination was metabolizing the hydrocarbons (FIG. 4).

### Example 3

### RAPD-PCR DNA Profiles

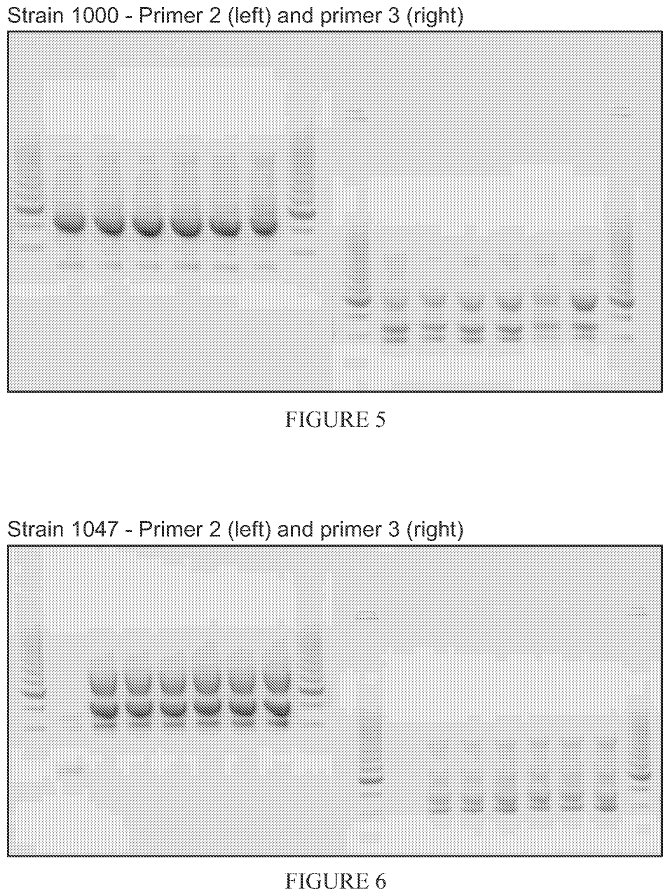

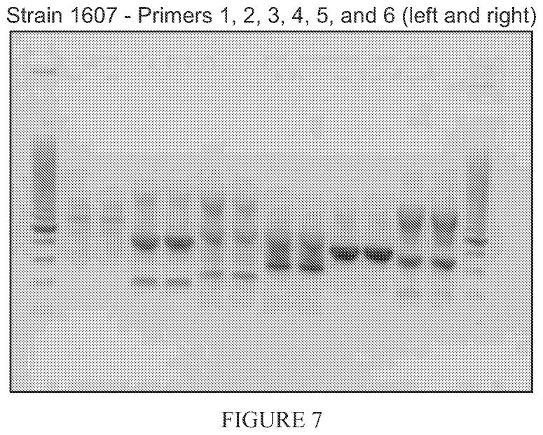

The Randomly Amplified Polymorphic DNA PCR method (herein referred to as RAPD-PCR) was used to identify genetic variability of each strain. Preparation of the DNA to be used in the RAPD-PCR reaction was done by using the QIAGEN® Tissue and Blood single column kit (QIAGEN®, Venlo, The Netherlands). FIGS. 5 to 7 illustrate RAPD-PCR results for the strains described in this application. The results show that the strains are unique from each other.

1. A method of treating water and/or soil by contacting the water and/or the soil with a Bacillus strain to remove a hydrocarbon, the method comprising contacting the water and/or the soil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the hydrocarbon.

2.-3. (canceled)

4. The method of claim 1 wherein the hydrocarbon is a short-chain to medium-chain hydrocarbon.

5. The method of claim 1 wherein the hydrocarbon is selected from the group consisting of toluene, benzene, kerosene, and combinations thereof.

6. The method of claim 1 wherein the hydrocarbon is selected from the group consisting of a gas range organic (GRO), a diesel range organic (DRO), and an oil range organic (ORO).

7. The method of claim 1 wherein the hydrocarbon is a petroleum hydrocarbon.

8. The method of claim 1 wherein the hydrocarbon is a long-chain hydrocarbon.

9. The method of claim 1 wherein the Bacillus strain produces an enzyme selected from the group consisting of an oxidoreductase, a hydrolase, a transferase, a lyase, an isomerase, a ligase, a peroxidase, a laccase, an esterase, an amylase, a protease, a xylanase, a lipase, a cellulase, an oxygenase, a reductase, an oxidase, a hydroxylase, a dehydrogenase, and combinations thereof.

10. The method of claim 1 further comprising treating the water and/or the soil with another Bacillus strain.

11. The method of claim 1 wherein the effective amount of the Bacillus strain is
i) about 1.0×10² CFU/gram of the water and/or the soil to about 1.0×10⁶ CFU/gram of the water and/or the soil;
ii) about 1.0×10² CFU/gram of the water and/or the soil to about 1.0×10⁴ CFU/gram of the water and/or the soil; or
iii) about 1.0×10² CFU/gram of the water and/or the soil to about 1.0×10³ CFU/gram of the water and/or the soil.

12.-13. (canceled)

14. The method of claim 1 further comprising contacting the water and/or the soil with an exogenous enzyme.

15. A commercial package comprising isolated Bacillus strains wherein the Bacillus strains consist of Bacillus licheniformis MDG-1000 (NRRL No. B-67888), Bacillus pumilus MDG-1047 (NRRL No. B-67891), and Bacillus amyloliquefaciens MDG1607 (NRRL No. B-67666), or strains having all of the identifying characteristics of these strains.

16.-18. (canceled)

19. The commercial package of claim 15 further comprising a carrier for the Bacillus strains.

20. The commercial package of claim 15 wherein the Bacillus strains are in powder form.

21.-22. (canceled)

23. The commercial package of claim 15 further comprising a binder.

24. The commercial package of claim 23 wherein the binder is selected from the group consisting of clay, yeast cell wall components, aluminum silicate, and glucan, or combinations thereof.

25. The commercial package of claim 19 wherein the carrier is selected from the group consisting of a salt, mineral oil, a dextrin, whey, sugar, sucrose, limestone, yeast culture, dried starch, sodium silico aluminate, silicon dioxide, polypropylene glycol, polysorbate 80, vegetable oil, water, rice hulls, and combinations thereof.

26. A method of removing oil in a cleaning application by contacting the oil with a Bacillus strain to remove the oil, the method comprising contacting the oil with an effective amount of an isolated Bacillus strain selected from the group consisting of Bacillus strains Bacillus licheniformis MDG-1000 (NRRL No. B-67888), Bacillus pumilus MDG-1047 (NRRL No. B-67891), Bacillus amyloliquefaciens MDG1607 (NRRL No. B-67666), and strains having all of the identifying characteristics of these strains, and removing the oil.

27. The method of claim 26 wherein the cleaning application is an industrial cleaning application.

28. The method of claim 26 wherein the oil is from an oil stain on concrete.

29. The method of claim 26 wherein the oil is on metal.

30.-31. (canceled)
